(19) United States
(12) Patent Application Publication
(10) Pub. No.: US 2025/0050358 A1
(43) Pub. Date: Feb. 13, 2025

(54) SPRAYING HEAD

(71) Applicant: APTAR FRANCE SAS, LE NEUBOURG (FR)

(72) Inventor: Ludovic PETIT, VITOT (FR)

(73) Assignee: APTAR FRANCE SAS, LE NEUBOURG (FR)

(21) Appl. No.: 18/720,298

(22) PCT Filed: Dec. 15, 2022
(86) PCT No.: PCT/FR2022/052379

§ 371 Date: Jun. 14, 2024

(30) Foreign Application Priority Data
Dec. 17, 2021 (FR) FR2113871

(51) Int. Cl. B05B 1/34; A61M 11/04; B29C 33/00; B29L 31/00

(52) U.S. Cl. CPC B05B 1/3426; A61M 11/04; B29C 33/0033; B29L 2031/753

(57) ABSTRACT

Spraying head having a head body forming a housing having an axis X defining an insertion opening and a substantially flat internal wall through which a central spraying opening extends and a spray nozzle, substantially tubular in shape and axially engaged in the housing of the head body via the insertion opening, the spray nozzle having an inlet that opens into a central internal duct and an external wall defining a central cavity centred on the central spraying opening and a plurality of swirl channels that open tangentially into the central cavity and communicate with the central internal duct. The swirl channels extend axially beneath the central cavity as far as the central internal duct, so as to be in direction communication therewith.

The present invention relates to a spraying head intended to be associated with a dispensing member, such as a pump or a valve.

In the prior art, spraying heads are already known, comprising:
a head body forming a housing having an axis X defining an insertion opening and a substantially flat internal wall through which a central spraying opening extends,
a spray nozzle, substantially in the shape of a tube, axially engaged in the housing of the head body via the insertion opening, the spray nozzle comprising an inlet that opens into a central internal duct and an external wall defining a central cavity centred on the central spraying opening and a plurality of swirl channels that open substantially tangentially into the central cavity and communicate with the central internal duct.

Conventionally, the communication between the central internal duct and the tangential channels is made through radial side windows which extend radially outwards from the central internal duct. These radial side windows open onto axial connecting channels which are formed around the spray nozzle, for example in the form of flat spots. These axial connecting channels extend axially and join the swirl channels substantially at a right angle. Generally, the axial depth of the swirl channels is substantially equal to that of the central cavity, which will form the swirl chamber in engagement with the flat internal wall through which the central spraying opening extends. This, the fluid product travels through the central internal duct, then radially through the radial side windows, then axially through the axial connecting channels, then radially through the swirl channels, in order to arrive in the swirl chamber in which the fluid product forms a vortex and is finally dispensed through the central spraying opening in the form of a cloud of fine droplets. Usually, there are two or three radial side windows and as many axial connecting channels and swirl channels.

To mould radial side windows, compartment pins, i.e. moving transversally (perpendicularly) with respect to the axis X of the housing. The implementation of these compartment pins is always difficult, and therefore a source of problems.

The aim of the present invention is to simplify the moulding of such a spraying head by removing the compartment pins. Another aim is to produce a moulding assembly which only comprises two parts which can be axially moved against one another.

To do this, the present invention proposes that the swirl channels extend axially beneath the central cavity as far as the central internal duct, so as to be in direct communication therewith. In other words, it can be considered that the conventional swirl channels, the depth of which is equal to that of the central cavity, in this case are extended axially downwards as far as meeting the central internal duct.

The invention can also be defined as follows: the swirl channels extend partially into the axial extension of the central internal duct, so as to define axial fluid passages, which extend from the central internal duct as far as the external wall. Advantageously, the swirl channels comprise outlets in the central cavity which axially communicate with the central internal duct.

According to an advantageous embodiment of the invention, the central internal duct can comprise a frustoconical portion, the swirl channels communicating directly with this frustoconical portion through windows which are inclined with respect to the axis X. To achieve these inclined windows, a moulding assembly can be used according to the invention, comprising a first axial pin to form the central internal duct and a plurality of second axial pins to form the swirl channels, the first pin and the second pins having conical surfaces, intended to come into mutual cone-to-cone contact by relative axial movement, so as to form the inclined windows. It must be noted that a cone-to-cone contact makes it possible to avoid any friction of edges of pins during mould release. Naturally, the cone-to-cone contact also makes it possible to avoid any sliding contact of pins against one another.

According to another aspect of the invention, the swirl channels can each comprise main walls which extend from the external wall as far as a bottom which is located axially at the central internal duct and which communicates with the central internal duct through a window, the two main walls forming an angle between them by moving radially closer to the central cavity. Advantageously, the central cavity can be axially separated from the windows by a stopper located axially downstream from the central internal duct.

Relating to the dimensional aspects, the swirl channels can have an axial depth which is 3 to 10 times greater, and advantageously 5 to 10 times greater, than that of the central cavity. On the other hand, the windows can have an axial height which represents around half of the axial depth of swirl channels.

The present invention also defines a fluid product dispenser comprising a dispensing member, such as a pump or a valve, mounted on a fluid product reservoir, and a spraying head mounted on the dispensing member.

Beyond the moulding and mould release facility that the invention provides, other advantages have been seen, like for example the spray angle, which is less open or tighter with respect to standard heads. In a nasal application, a better deposit is obtained in the olfactory zone, which is located quite far into the nasal cavity. Indeed, as the spray is more axial, it penetrates more deeply into the nose. Moreover, given that the flow is more axially central, without radial baffle, the fluid load losses are decreased, and the flow rate is better and favours an at least equivalent spray quality for a lower provided pressure. Therefore, a pump can be used, which provides a lower pressure. The quality of the spray is also improved, while preserving a known and standardised swirl chamber profile. In the spray, the size of the drops is more uniform for a lower pump pressure, enabling a lower actuation speed and lower force for an at least equivalent result in the quality of the spray. Finally, it has also been observed that the reduced load loss makes it possible to decrease the dose variation, and therefore to increase the consistency of the doses dispensed.

The spraying head of the invention has an advantageous application as a nasal spray, but it can be used in other pharmaceutical or other fields, like cosmetics and perfumery.

The spirit of the invention resides in the axial travelling of the fluid product from the axial central housing through the swirl channels as far as the periphery of the swirl chamber. Graphically, a needle can be introduced axially into a swirl channel in the proximity of the central cavity, this needle penetrating into the central internal duct without deviating from its axial orientation. The formation of direct connection windows, disposed in an inclined manner, between the central internal duct and the swirl channels, enables this axial communication. A cone-to-cone contact moulding assembly makes it possible to form these oblique or inclined windows.

The invention will now be more fully described in reference to the accompanying drawings, giving as a non-limiting example, an embodiment of the invention.

In the figures:

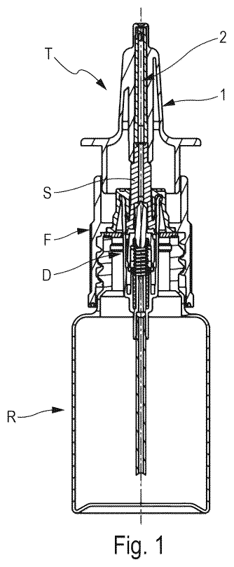

FIG. 1 is a cross-sectional view through a fluid product dispenser integrating a spraying head according to an embodiment of the invention,

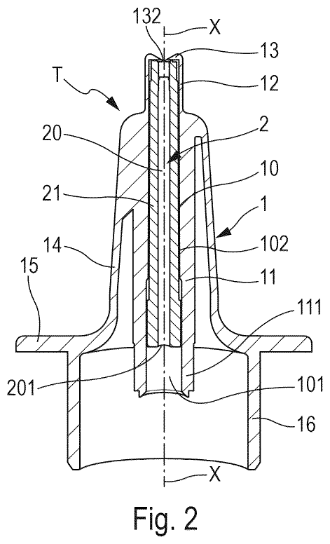

FIG. 2 is a greatly enlarged view of the spraying head of FIG. 1,

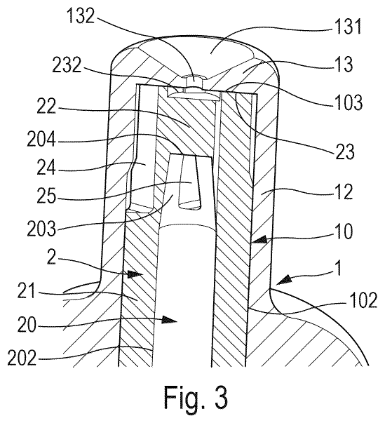

FIG. 3 is an even more greatly enlarged view of the upper portion of the spraying head of FIG. 2,

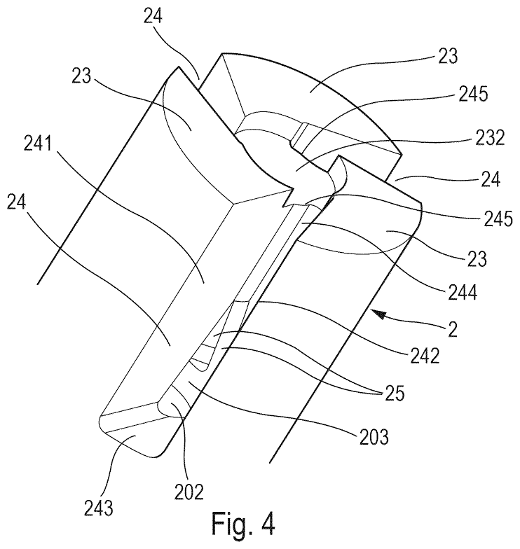

FIG. 4 is a very greatly enlarged view of the upper portion of the spray nozzle of the spraying head of FIGS. 1 to 3,

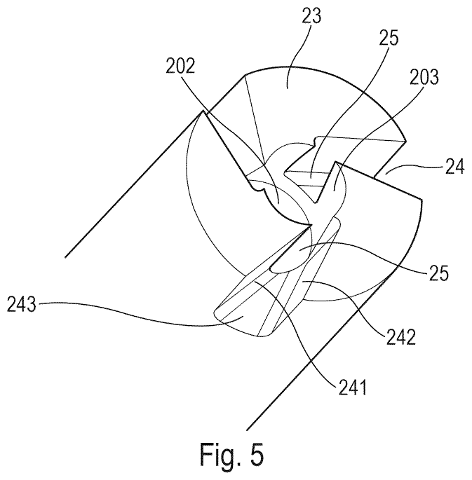

FIG. 5 is a view similar to that of FIG. 4, but with a cut-away portion,

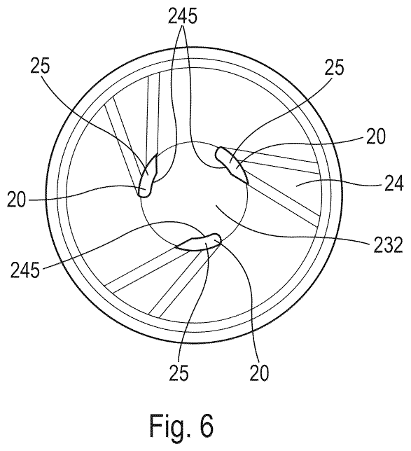

FIG. 6 is a top axial view of the spray nozzle of the spraying head of the invention,

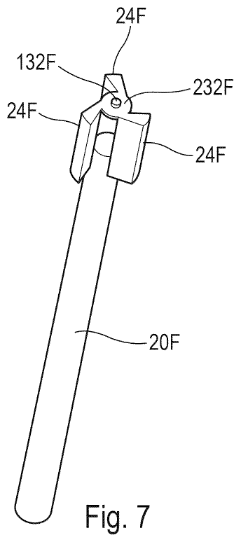

FIG. 7 illustrates the fluid volume inside the spraying head of the invention, and

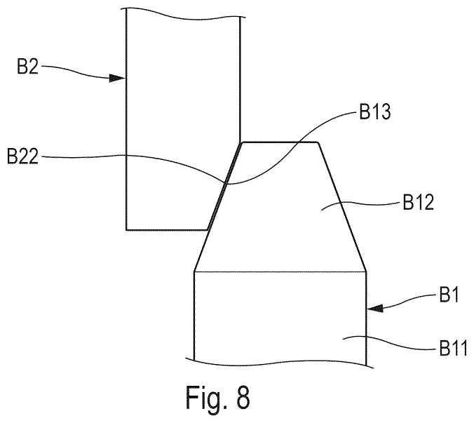

FIG. 8 is a vertical cross-sectional schematic view illustrating a moulding assembly making it possible to mould the spray nozzle of the spraying head of the invention.

In FIG. 1, a fluid product dispenser is seen, comprising a fluid product reservoir R on which there is mounted a dispensing member D, which can be a pump or a valve. The dispenser also comprises a fixing member F which firmly and sealingly holds the dispensing member D on the fluid product reservoir R. This fixing member F can, for example, be screwed on a threaded neck of the reservoir R. Other fixing techniques are also possible. The dispensing member D comprises a valve rod S which is able to be axially moved back and forth against a return spring. The fluid product pressurised in the dispensing member D is discharged through this valve rod S. A spraying head T is mounted on the free end of the valve rod S. This spraying head aims to convey the fluid product coming from the valve rod S to a spraying opening 132, at which the fluid product is dispensed in the form of a spray of fine droplets. The dispensing head T also serves as a pushbutton making it possible to move the valve rod S.

This design is entirely conventional for a fluid product dispenser in the pharmacy field, but also those of cosmetics, perfumery, and even food.

In FIG. 2, it can clearly be noted that the dispensing head T is designed, in this case, for a nasal application, making it possible to spray a fluid product into the nasal cavity through a nostril of the nose. This particular application must not be considered as limiting for the invention: other spray head forms, designed for other applications, can be envisaged without moving away from the scope of the invention.

The spraying head T first comprises a head body 1, which can be made of one piece by injection-moulding an appropriate plastics material. The head body 1 comprises an axial sheath 11 which forms, at its lower end, a connection sleeve 111 and which is extended at its upper end by an end tip 12 which forms a disc 13 pierced by a central spraying opening 132. As can be seen in FIG. 1, the spraying head T is mounted on the dispensing member D by sealed fitting of the sleeve 111 around the free end of the valve rod S.

The sheath 11 and the tip 12 internally defining a housing 10 defining an insertion opening 101, an internal wall 102 substantially cylindrical in shape and a substantially flat upper internal wall 103. In FIG. 3, it can be noted that the flat internal wall 103 forms the lower face of the disc 13 which closes the end tip 12. The upper opposite face of the disc 13 forms a diffuser cone 131 centred on the spraying opening 132.

Thus, the housing 10 can have a relatively simple configuration, practically tubular in shape, about a longitudinal axis X, with an open lower end and a closed upper end with a spraying opening 132.

The head body 1 also forms an outer casing 14 which extends downwards from the junction between the sheath 11 and the tip 12. This outer casing 14 can have a frustoconical shape to facilitate the insertion of the head body 1 inside a nostril. At its lower end, the outer casing 14 is extended through an annular flange 15 which serves as a pushbutton: the user can thus use their index finger and their middle finger to press on the annular flange 15 and thus actuate the dispenser by depression of its valve rod S. Finally, the head body 1 comprises a skirt 16 which extends downwards from the annular flange 15. This skirt 16 has a technical guiding function, as well as an aesthetic function consisting of masking the valve rod S.

The spraying head T also comprises a spray nozzle 2, which can also be made of one piece by injection-moulding an appropriate plastics material. The spray nozzle 2 has a substantially tubular shape, about the axis X, so as to be able to be axially engaged inside the housing 10 through the insertion opening 101. The spray nozzle 2 thus forms a tubular body 21 internally defining a central internal duct 20 with an inlet 201 which opens into the insertion opening 101 of the head body 1. The central internal duct 20 extends inside the tubular body 21 as far as a stopper 22 which axially blocks it.

Externally, the spray nozzle 2 forms an external wall 23, which is opposite the inlet 201. This external wall 23 defines a central cavity 232 which is centred on the spraying opening 132. The cavity 232 and the internal wall 103 thus together define a swirl chamber axially centred on the spraying opening 132. The central cavity 232 defines a bottom, advantageously flat, and side wall segments, which connect the bottom to the external wall 23. The external wall 23 also forms a plurality of swirl channels 24 which advantageously open tangentially into the central cavity 232, between side wall segments 232. This can be seen more clearly in FIG. 4. A swirl system formed of a central chamber supplied by tangential channels is an absolutely conventional design.

According to the invention, the swirl channels 24 extend axially beneath the central cavity 232, around the stopper 22, and even beneath the stopper 22 so as to reach the central internal duct 20. More specifically, the swirl channels 24 communicate directly with a frustoconical portion 203 of the central internal duct 20 through the windows 25. Given that the frustoconical portion 203 is appropriately frustoconical, the windows 25 extend in an inclined manner with respect to the longitudinal axis X of the spray nozzle 2. It can even be noted that the windows 25 have a trapezoidal shape with a wide base and a restricted top.

By referring to FIGS. 4 and 5, it can be seen that each swirl channel 24 comprises two main walls 241 and 242, advantageously flat which extend axially from the flat wall 23 downwards as far as a bottom 243 at the stopper 22. The channel 24 also forms an outlet wall 244 which forms the axial junction between the two main walls 241 and 242. It can also be noted that these two main walls 241 and 242 extend with an angle so as to move closer in the direction of the central cavity 232 and of the windows 25. The outlet walls 244 can be profiled and extend axially from the central cavity 23 to the windows 25.

In FIGS. 4 and 6, it can be noted that the channels 24 define outlets 245 in the central cavity 232, these outlets 245 being formed above the outlet walls 244 which extend axially above the inclined windows 25. Thus, as can be seen more specifically in FIG. 6, the channels 24 at their outlets 245 or their outlet walls 244 communicate axially directly with the central internal duct 20 through the inclined windows 25. It can be noted that the central cavity 232 is not fully circular, since interrupted by the outlets 245 or the outlet walls 244, which can be shaped. It can also be said that the bottom of the central cavity 232 defines external edges at the level of its junction with the outlet walls 244, which plunge axially into the central internal duct 20 through the inclined windows 25. Thus, the outlets 245 are located axially above the central internal duct 20.

It must be noted that the formation of inclined windows 25 is only one preferred embodiment of the invention, since it would also be possible to produce fully axial windows with swirl channels 24 which would come into flush contact with a fully cylindrical internal duct. The implementation of inclined windows 25 is particularly advantageous, given that the axial communication is greater and that the moulding of the spray nozzle 2 is facilitated.

Concerning the dimensions of the spray nozzle 2, it can be said that the axial depth of the swirl channels 24 is considerably greater than that of the central cavity 232, around three to ten times, and advantageously five to ten times. It can also be noted that the axial height of the windows 25 corresponds to around half of the axial depth of the channels 24. The height of the shaped wall 244 corresponds to around the height of the windows 25. The diameter of the central cavity 232 corresponds to around the diameter of the cylindrical portion 202 of the central internal cavity 20.

FIG. 7 illustrates the volume that the fluid product can occupy inside the spraying head T of the invention. The rod 20F represents the internal volume of the central duct 20. This volume 20F communicates directly and axially with three volumes 24F representing the swirl channels 24. These volumes 24F are presented in the form of substantially triangular cross-sectional slices. The volume 20F is separated from the volume 232F, which corresponds to the volume of the swirl chamber 232, due to the presence of the stopper 22. The volumes 24F, corresponding to the swirl channels 24, communicate with the volume 232F so as to form a type of helix. Finally, the volume 132F corresponds to the volume of the spraying opening 132. Again, it is understood that the fluid product has a fully axial travelling between the volume 20F and the volume 232F through the volumes 24F which extend around the volume 20F and the volume 232F.

FIG. 8 very schematically and partially shows a moulding assembly making it possible to mould the spray nozzle 2. This moulding assembly comprises a first pin B1 intended for the formation of the axial internal duct 20. Due to this, the pin B1 first comprises a cylindrical portion B11 which ends via a frustoconical portion B12. Due to its conicity, the frustoconical portion B12 defines a conical wall B13. On the other hand, the moulding assembly comprises a plurality of second pins B2 intended for the formation of the tangential swirl channels 24. These pins B2 each define a conical surface B22, which comes, during the moulding in cone-to-cone contact on the surface B13 of the first pin B1.

This moulding assembly has a plurality of advantages. First, it is well-known that a cone-to-cone contact is easier to produce than a shearing sliding contact between two surfaces. Second, this cone-to-cone contact makes it possible to produce inclined windows 25 which increase the axial communication between the central internal duct 20 and the swirl channels 24.

In this embodiment, which corresponds to a nasal use, the housing 10 and the central internal duct 20 have a significant length. Other embodiments could very well be envisaged with a housing 10 and a duct 20 of reduced length, even very greatly reduced length. On the other hand, the embodiment which has been used to illustrated the invention comprises three swirl channels, but this number can very well be reduced to two or, on the contrary, be increased to four, even more.

Thanks to the invention, a spraying head with a central duct and swirl system with a direct axial supply between the central duct and the swirl system can be achieved.

1. A spraying head intended to be associated with a dispensing member, such as a pump or a valve, the spraying head comprising:
a head body forming a housing having an axis X defining an insertion opening and a substantially flat internal wall through which a central spraying opening extends,
a spray nozzle, substantially tubular in shape, axially engaged in the housing of the head body via the insertion opening, the spray nozzle comprising an inlet that opens into a central internal duct and an external wall defining a central cavity centred on the central spraying opening and a plurality of swirl channels that open tangentially into the central cavity and communicate with the central internal duct,
characterised in that the swirl channels extend axially beneath the central cavity as far as the central internal duct, so as to be in direction communication therewith.

2. The spraying head according to claim 1, wherein the swirl channels extend partially into the axial extension of the central internal duct, so as to define axial fluid passages, which extend from the central internal duct as far as the external wall.

3. The spraying head according to claim 1, wherein the swirl channels comprise outlets in the central cavity which communicate axially with the central internal duct.

4. The spraying head according to claim 1, wherein the central internal duct comprises a frustoconical portion, the swirl channels communicating directly with this frustoconical portion through windows which are inclined with respect to the axis X.

5. The spraying head according to claim 4, wherein the central cavity is separated axially from the windows by a stopper located axially downstream from the central internal duct.

6. The spraying head according to claim 1, wherein the swirl channels each comprise main walls which extend from the external wall as far as a bottom which is located axially at the central internal duct and which communicates with the central internal duct through a window, the two main walls forming an angle between them by moving radially closer towards the central cavity.

7. The spraying head according to claim 1, wherein the swirl channels each comprise main walls forming an angle between them by moving radially closer towards the central cavity, the main walls extending axially from the external wall into the central internal duct at the point where the main walls are closest together.

8. The spraying head according to claim 1, wherein the swirl channels each comprise two main walls, which are connected together by an outlet wall, which is extends axially from the central cavity into the central internal duct.

9. The spraying head according to claim 8, wherein the swirl channels comprise outlets in the central cavity, which are formed in the axial extension of the outlet walls.

10. The spraying head according to claim 1, wherein the swirl channels have an axial depth which is 3 to 10 times greater, and advantageously 5 to 10 times greater, than that of the central cavity.

11. The spraying head according to claim 4, wherein the windows have an axial height which represents around half of the axial depth of swirl channels.

12. A fluid product dispenser comprising a dispensing member, such as a pump or a valve, mounted on a fluid product reservoir, and a spraying head, according to claim 1, mounted on the dispensing member.

13. A moulding assembly to mould a spray nozzle (2) according to claim 1, comprising a first axial pin to form the central internal duct and a plurality of second axial pins to form the swirl channels, the first pin and the second pins having conical surfaces intended to come into mutual cone-to-cone contact by relative axial movement.
